(19) United States
(12) Patent Application Publication
(10) Pub. No.: US 2011/0165639 A1
(43) Pub. Date: Jul. 7, 2011

(54) REFINERY PROCESS TO PRODUCE BIOFUELS AND BIOENERGY PRODUCTS FROM HOME AND MUNICIPAL SOLID WASTE

(75) Inventors: Miguel Ascon, Baltimore, MD (US); Dolores Ascon, Baltimore, MD (US)

(73) Assignee: BriJen Biotech, LLC, Baltimore, MD (US)

(21) Appl. No.: 13/059,173

(22) PCT Filed: Aug. 17, 2009
(86) PCT No.: PCT/US2009/053984

§ 371 Date: Feb. 15, 2011

Related U.S. Application Data
(60) Provisional application No. 61/189,037, filed on Aug. 15, 2008.

(51) Int. Cl. C12P 7/64; C12P 1/00; C12P 5/02; C12P 3/00; C12P 7/10; C12P 7/16; C12P 7/04; C12M 1/00

(52) U.S. Cl. 435/134; 435/41; 435/167; 435/168; 435/165; 435/160; 435/157; 435/292.1

(57) ABSTRACT

The present invention provides methods of a process (SOLWASFUEL system) for producing biofuel and bioenergy products using, as starting raw material, home and municipal organic solid waste, including recalcitrant lignocellulosic materials of paper, cardboards, organic plastics, cellulose plants, and food waste. In accordance with the invention, home and municipal solid waste, can serve as a carbon source to support the metabolism of synthetic microorganisms to produce biofuels and bioenergy products.

### PRIORITY

This application claims priority to U.S. Provisional Application No. 61/189,037, filed Aug. 15, 2008, which is hereby incorporated by reference in its entirety.

### FIELD OF THE INVENTION

The present invention relates a process for the production of biofuels and bioenergy products, including biodiesel, ethanol, butanol, methane, hydrogen, and methanol among others. The present invention relates to the production of such biofuels from home and municipal solid waste, particularly all organic materials, including cellulosic, hemicellulosic, lignocellulosic, as well as all amilolytic, proteinic and lipidic components of food waste materials.

### BACKGROUND OF THE INVENTION

There is an ever-increasing demand for renewable biofuels and bioenergy products as an alternative to fossil fuels. Biofuels are currently produced from, for example, various cellulosic materials and sugar-based plants, including sugarcane, beets, corn, rice, potatoes (among others), as well as wood chips. While the process is straight forward, producing biofuels and bioenergy products from these materials is, overall, inefficient and expensive given the cost of the source materials, and tends to drive up the price of food. Further, the current raw material sources for production of biofuel will not be sufficient to meet the escalating demands.

Municipal Solid Waste (MSW) include wastes from residential, multifamily, commercial and institutional (e.g. schools, government offices) sources. The U.S. Environmental Protection Agency (EPA) defines MSW as including “durable goods, non-durable goods, containers and packaging, food wastes and yard trimmings, and miscellaneous inorganic wastes.” MSW, otherwise known as trash or garbage, consists of everyday items such as product packaging, grass clippings, furniture, clothing, bottles, food scraps, newspapers, appliances, and batteries. The definition does not include all forms of solid waste, such as construction and demolition debris, industrial process wastes, and sewage sludge.

Under current policy, the Energy Information Administration (EIA) differentiates between biogenic and nonbiogenic waste in MSW, with biogenic waste excluding plastics, metals, rubber, and other nonorganic material. As of 2005, approximately 63 percent of the waste stream by weight was considered biogenic. This accounted for roughly 56 percent of the total energy content of managed MSW (167 trillion Btu). Although it is clearly that the entire waste stream should be treated as a renewable feedstock, only biogenic waste may used to produce biofuels.

254.1 million tons of MSW were generated in 2007, similar to the amount generated in 2006. Of this, 63.3 million tons were diverted to recycling, 21.7 million tons were diverted to composting, and 31.9 million tons were combusted with energy recovery. The remaining 137.2 million tons were sent to landfills. The breakdown, by weight, of product categories generated in MSW in 2007 show that containers and packaging (paper and paperboard) comprised the largest portion of products generated in MSW, at about 31 percent (78.4 million tons). Nondurable goods were the second-largest fraction, at 24.5 percent (62.2 million tons). The third-largest category of products is durable goods, which made up 17.9 percent (45.4 million tons) of total MSW generation.

The highest rates of recovery as recycling in 2007 were achieved with yard trimmings, paper and paperboard, and metals. About 64 percent (20.9 million tons) of yard trimmings was recovered for composting or mulching in 2007. This represents a five-fold increase since 1990. Over 54 percent (45.2 million tons) of paper and paperboard was recovered for recycling in 2007. Recycling these organic materials alone diverted 26 percent of municipal solid waste from landfills and combustion facilities. In addition, about 7.2 million tons, or about 35 percent, of metals were recovered for recycling.

Landfills serve as a repository for biogenic carbon that may be biodegraded to methane and subsequently recovered for energy, or sequestered as not all biogenic carbon biodegrades in landfills (US EPA, 2006). Previous measurements of carbon sequestration and methane yield used individual waste components or mixed refuse but did not rigorously address the actual composition of waste discarded in landfills. Therefore, the damage that the methane and other toxic gases liberated from the buried waste landfills can cause to the atmosphere are not really known.

The per capita generation of MSW has remained relatively steady since 2000, when it peaked at 4.65 lbs/day, and at 2007 was estimated around 5 lbs/day. The per capita discard rate (the amount of trash sent to landfills after recycling, composting, and energy recovery) has remained virtually fixed at 2.5 lbs/day since 1960. This means that virtually the entire increase in individual waste generation has been treated in ways other than landfilling. Regardless, the total amount of MSW generated is expected to continue rising in the foreseeable future as a result of population growth.

Considering the following facts: a) the hutch amount of MSW produced with increasing rates, b) the biogenic nature of this waste to be recovered and reused, c) the non efficient methods actually used to disposal, and e) the negative effects caused in the environment and health of population. These facts, convert the biogenic MSW as the more attractive resources to be used as raw materials to produce biofuels and bioenergy products to supply the energetic needs in the all world.

Here we describe a refinery process that could will used for recovering valuable components of biogenic home and municipal solid waste. This waste mainly include recalcitrant cellulosic, hemicellulosic, lignine materials, as well as soft food waste from home and restaurants. This process could help satisfy the energy needs of countries, while simultaneously reducing the impact of such waste on the environment and the health of the population.

### SUMMARY OF THE INVENTION

The present invention provides methods and systems for producing biofuel and bioenergy products using, as starting raw material home or municipal solid organic waste. In accordance with the invention, home and municipal solid waste, including all organic components, can serve as a carbon source to support the metabolism of fermentative, methogenic, and/or photosynthetic microorganisms to produce biofuels and bioenergy products.

The hutch amount of MSW (around 254.1 million tons) produced is not correctly treated, nothing well disposed, conducing to a negative impact in the environment and health of population. For example, the recalcitrant cellulosic compounds are not totally recycled and most are disposed in landfills to generate contamination to the atmosphere. Also the food waste generated at home or restaurants are disposed improperly to waste water or landfill systems. In accordance with the invention, input solid waste materials (recalcitrant cellulosic materials) are subjected to pre-treatment with physicochemical process to hydrolyze the cellulolytic materials and to extract the basic basic carbon sources, followed by the biosynthesis of biofuel and bioenergy products from this organic material. The biofuel or bioenergy products may include ethanol, methanol, butanol, biodiesel, methane, and hydrogen, among others.

The production process of biofuels and bioenergy products from solid wastes, could in this invention, be used at several scales. For example, It could be used efficiently at home micro-refinery scale to transform all domestic garbage into valuable fuels including gas and biodiesel, among others. Also could be used at pilot scale to treat all garbage produced in the restaurants, hotels, and hospitals. But also this invention process could be used to produce biofuels and bioenergy products from MSW at industrial macro-refineries.

In one aspect, the present invention provides a method for generating one or more biofuels or bioenergy products using home or municipal solid waste as raw materials. All organic materials from solid waste mainly including cellulosic residues and food waste are pre-treated using physicochemical methods including acid/alkaline compounds, and high temperatures. The solid biomass “treated” becomes uniform standardized and becomes a suitable carbon source for the synthesis of one or more biofuels from the organic material by fermentative and/or methanogenic microorganisms.

The home or municipal solid waste previously pre-treated is fed, or circulates to, one or more bioreactors for the biosynthesis of bioenergy products including, for example, methane, ethanol, butanol, and methanol, which are recovered and/or purified. For example, ethanol, butanol, or methanol may be produced by anaerobic fermentation of the organic material by one or more microorganisms such as certain bacteria, yeasts, and filamentous fungi, and the biofuel products are subsequently recovered. Alternatively, or in addition, methane may be produced by one or more methanogenic microorganisms (such as a microorganism consortium), and recovered and/or purified. Unprocessed organic material is subjected to further treatment, for example, by feedback through the system.

The CO₂, as produced during the anaerobic processes, may be captured before escape to environment and used as a carbon source to support the growth and metabolism of photosynthetic microorganisms (e.g., blue-green algae) to synthesize additional biofuels, such as biodiesel (or synthetic intermediates such as lipids) and hydrogen gas.

In a second aspect, the present invention provides systems for generating biofuels and bioenergy products, for example, in accordance with the methods described herein. The system may be an integrated system for coupling the solid waste pre-treatment process with the biosynthesis of bioenergy products. Alternatively, the system may comprise a pre-treat waste system, and a separate biosynthesis system. For example, the suitable carbon source for biosynthesis may be produced in a pre-treated bioreactor system, and the resulting biomass (treated waste) subsequently fed to the biosynthesis system. Such systems allow for the transportation of waste treated at one location, to be transported to another for synthesizing biofuel or bioenergy products. The system may be connected to, or positioned or located near, the production or source of sludge, so as to obviate the need to transport the waste material for treatment or disposal.

The system comprises one or more bioreactors suitable for pre-treatment of home solid waste by the action of physicochemical conditions to produce treated or processed sludge. For example, the pre-treatment system may comprise at least one continuous stirred-tank reactor, and/or at least one tubular reactor, such as a recirculating tubular reactor. In some embodiments, the pre-treatment system comprises a continuous stirred-tank reactor followed by a tubular plug flow reactor, thereby providing the mixing necessary to support the hydrolysis of cellulose materials. The system may further comprise a sieve or strainer for separating liquid of solid phases of waste, such that liquid waste may be fed to synthetic bioreactor as a carbon source for biofuel production and solid waste feedback to further pre-treatment.

The system further comprises tubular bioreactor(s) suitable for the biosynthesis of biofuels from the pre-treated biomass. The bioreactor(s) for biosynthesis contain naturally selected or genetically engineered microorganisms for the synthesis of products such as, for example, ethanol, methanol, butanol, and methane from pre-treated organic solid waste. The biosynthesis reactor(s) may be anaerobic multiphasic bioreactor(s) having fermentative and/or methanogenic microbes forming biofilms on solid surfaces.

In other embodiments, the biosynthesis system further comprises at least one photo bioreactor connected to the anaerobic reactor to support the production of additional biofuel products including the hydrogen and biodiesel by photosynthetic microorganisms, including one or a consortium of algae(s). The metabolism of the photosynthetic microorganisms is supported by the CO₂ produced during anaerobic biosynthesis, and also by luminosity of natural or artificial bright light.

The system may further comprise mechanism(s) for separating, collecting, and/or recovering bioenergy products resulting from the biosynthesis bioreactors. The system may further comprise a container or feed to recover non-fuel compounds for feedback to the bioleaching system. Thus, the system may comprise a feedback connection between the biosynthesis system and the pre-treated system to continuously recycle all materials not completely used, to avoid the liberation of polluted materials to the environment.

Thus, in accordance with this invention, all organic solid waste produced at municipal range or home may directly used to produce valuable biofuels including ethanol, butanol, methane, biodiesel, and hydrogen among others. Avoiding in this way the incorrect disposal of the waste and the contamination of the environment.

### DESCRIPTION OF THE FIGURES

FIG. 1 is a flow diagram illustrating the method and system (SOLWASFUEL) of the invention. Cellulosic materials from home and municipal solid waste are pre-treated in a physicochemical reactor to hydrolyze the cellulosic, hemicellulosic and lignine materials. The treated solid waste is used as an available carbon source in a heterogeneous bioreactor to synthesize and produce biofuels. Remaining material is fed back through the system for further treatment and biosynthesis.

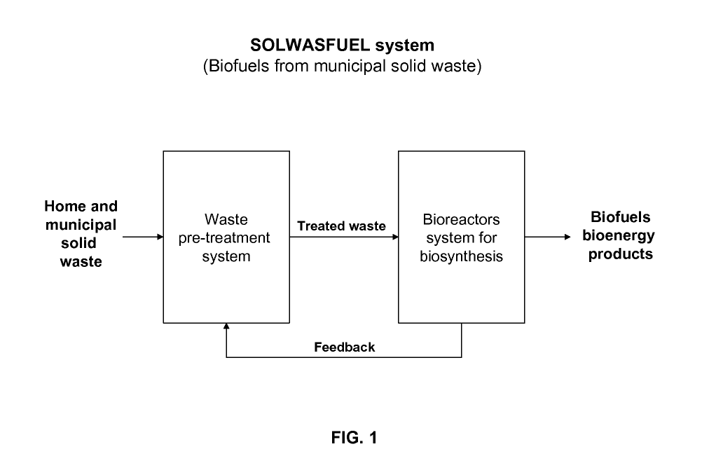

FIG. 2 illustrates an exemplary integrated system and process of the invention. The pre-treating system comprises mainly of a physicochemical reactor to hydrolyze all cellulosic solid waste materials, and also of a stabilized reactor where the hydrolyzed materials and soft food waste are stocked. The heterogeneous bioreactors for biosynthesis may comprise anaerobic bioreactor(s) and photo reactor(s). The pre-treatment and the biosynthesis processes/systems are connected by a feedback loop, such that untreated waste and metabolites are recycled through the system.

FIG. 3 illustrates each reactor/bioreactor in the exemplary integrated system. The pre-treat reactors are equipped of a agitated system to mixing the waste during the treatment. The biosynthesis reactors contain a heterogeneous fluidized bed to reach high conversion rates to product. A photosynthesis bioreactor is supported by the CO₂ emission from the anaerobic fermentation reactor.

FIG. 4 illustrates the structure of the fluidized biofilm pellets formed in the heterogeneous bioreactors. These bioreactors each have solid or liquid surfaces, such as porous glass, silicone rubber, silicone oil, among others, to support microbial biofilm pellets. Such heterogeneous bioreactors maximize surface areas to support extensive microbial metabolism. This kind of bioreactors may contain, in addition to solid support surfaces, liquid surfaces as well as cellular, aqueous, and gas phases.

FIG. 5 shows the performance of a pre-treatment reactor to hydrolyze cellulosic materials from solid waste. After an 8-hours incubation period the hydrolysis using acid, alkali and alkali/peroxide reached until 58, 63, and 67%, respectively. In contrast, cellulose hydrolysis in a control system (without physicochemical treatment) was only around 4%.

FIG. 6 shows the performance of a heterogeneous anaerobic bioreactor containing a methanogenic consortium in the production of methane from treated and non-treated municipal solid waste. At the hydraulic retention time of 4 to 8 hours, approximately 310, 386, and 382 ml of biogas/gram of COD were produced from treated, non-treated, and half-treated, respectively. These results were compared with positive (ethanol) and negative (non-cellulose hydrolyzed) controls where around 400 and 56 ml gas/g of COD, respectively.

FIG. 7 shows the performance of a heterogeneous anaerobic bioreactor, containing a yeast culture, in the production of ethanol from treated and non-treated municipal solid waste. After 48 hours of culture, approximately 0.48, 0.52, and 0.54 ml of ethanol/gram of COD were produced from treated, non-treated, and half-treated, respectively. These results were compared with positive (glucose) and negative (non-cellulose hydrolyzed) controls where around 0.56 and 0.09 ml ethanol/g of COD, respectively.

FIG. 8 shows the performance of a heterogeneous photosynthetic bioreactor, containing a lipidic microalgae culture, in the production of lipids for biodiesel from CO₂ outlet from an anaerobic bioreactor. Performance was assessed by the yields of biomass according to the concentration of CO₂ injected. Substantial biomass is achieved at 1% and 0.5% CO₂ concentrations, with more biomass produced at 1%. The lipids extracted to produce biodiesel reached approximately 61% of the biomass produced at different concentration of CO₂.

FIG. 9 shows the performance of a heterogeneous bioreactor, containing a blue-green microalgae, in the production of hydrogen in relation to biomass (nmol hydrogen per gram of protein). The hydrogen release was approximately 164 nmol of hydrogen per gram of protein when biomass was grown at 1% of CO₂, while 87 nmol of hydrogen per gram of protein was produced when 0.5% CO₂ was injected.

### DETAILED DESCRIPTION OF THE INVENTION

The present invention provides the description of a process for producing biofuel and bioenergy products using, as starting raw material, municipal and home solid organic waste, including mainly the cellulose and food waste materials. In accordance with this invention, home or municipal solid waste may be treated and be used as a carbon source to support the biosynthetic metabolism of microorganisms in bioreactors to produce biofuels and bioenergy products.

### Home and Municipal Solid Waste

The present invention provides a refinery process to produce biofuel and bioenergy products using, as starting raw material, residential and municipal solid waste. Depending of the natural composition, in some embodiments, the organic MSW as food waste may be directly processed in a bioreactor to produce biofuels and bioenergy products. In other embodiments, the recalcitrant organic MSW including the lignocellulosic materials may be pre-treated before it can be bioprocessed in anaerobic bioreactors. The pre-treatment processes could include some chemical or physical processing such as cutting, breaking, acid/alkali treatment, mixing and thermal processing.

Sources of MSW, as characterized in this report, include both residential and commercial locations. We estimated residential waste (including waste from multi-family dwellings) to be 55 to 65 percent of total MSW generation. Commercial waste (including waste from schools, institutions, and businesses) constitutes between 35 and 45 percent of MSW. Local and regional factors, such as climate and level of commercial activity, contribute to these variations.

In some embodiments, MSW is the waste generated in a community with the exception of industrial and agricultural wastes. Hence MSW includes residential waste (e.g., households), commercial (e.g., from stores, markets, shops, hotels etc), and institutional waste (e.g., schools, hospitals etc). Paper, paperboard, garden and food waste can be classified in a broad category known as organic or biodegradable waste. The present invention provides methods and systems that are versatile with regard to synthesizing biofuel and bioenergy components from these recalcitrant solid waste.

The organic compound fraction of MSW in the US represents 70% of the waste composition and consists of paper, garden waste, food waste and other organic waste including plastics. The biodegradable fraction (paper, garden and food waste) accounts for 53% of waste composition. Therefore, treatment of these wastes is an important component of an integrated solid waste management strategy and reduces both the toxicity and volume of the MSW requiring final disposal in a landfill. Such waste may be converted to one or more biofuel/bioenergy products in accordance with the invention, while avoiding the impact of the contaminants on the environment, as well as avoiding the large costs of many specialized treatment systems.

The largest category of municipal solid waste (MSW) going to landfills is food waste consisting of food scraps from restaurants, produce markets, fish markets, school cafeterias, homes, and wherever else food is prepared. Food waste is high in energy potential and should be recovered, rather than being lost in a landfill. Waste food discarded in large amounts all across the US, and the world is biomass, and can be used in the biomass fermentation process to produce energy. With all of the food waste that is thrown away, the bioconversion process according this invention, would allow this waste to be reused instead, and become a source of energy for electricity, and fuel for your car.

Also, the fact that food waste is full of sugars, oils, and fats make this waste perfect for creating, in accordance with this invention, different kinds of biofuels and bioenergy products. Many restaurants throw out thousands of gallons of used cooking oil and frying fat every single day, and this represents a large amount of biodiesel fuel that could be created using the waste food and bioconversion technology. In addition, households represent 60% of all the waste food thrown out, and this equates to tons of table scraps and discarded food which could be used to heat your home, power your light and appliances, and fuel your vehicle as well.

### Basic Media

Generally, the municipal, industrial, and/or farm sewage sludge (as described above) will contain organic and inorganic nutrients for supporting microbial growth and metabolism, e.g., in an aqueous phase. However, where these inorganic nutrients are absent, or are present in insufficient amounts, the sludge-containing material may be supplemented with an aqueous phase containing a basic mineral salt medium to support microbial growth and metabolism of sludge-digesting or sludge-treating microorganisms.

Thus, in the bioreactors described below, the aqueous phase comprise inorganic nutrients to support microbial growth and metabolism. For example, the aqueous phase may comprise a mineral salts medium. Nitrogen and phosphorous are the main nutrients added to the aqueous phase. Micronutrients such as Ca, Zn, Mn, Cu, Fe, Mg, Mn, Mb, and S may also be present in at least trace amounts. An exemplary mineral salts medium is KHCO₃ (e.g., 2 g/L), NaHCO₃ (e.g., 1.8 g/L), KH₂PO₄ (e.g., 0.7 g/L), Na₂HPO₄.12H₂O (1.4 g/L), MgSO₄.7H₂O (e.g., 0.2 g/L), and (NH₄)₂SO₄ (e.g., 0.8 g/L). The medium may further contain trace elements as may be required to support growth and vitality of the microorganisms, such as Ca(H₂PO₄) (e.g., 40 mg/L), ZnSO₄. 7H₂O (5 mg/L), Na₂MoO₄.2H₂O (2.5 mg/L), FeSO₄.7H₂O (1 mg/L), MnSO₄.H₂O (1 mg/L), and CuSO₄ (0.6 mg/L). The nutrient medium may of course be adjusted based upon the metabolic requirements of the microorganisms present for metal leaching and biosynthesis.

Generally, to support the physicochemical pre-treatment of recalcitrant MSW containing lignocellulosic materials, the presence of acid or alkali compounds (H₂SO₄ or NaOH) are necessary. For example, these chemical compounds may be used at concentrations in the range about 2 to 4% the H₂SO₄, and 0.5 to 6% the NaOH, depending of the natures of cellulosic material, and the time and incubation temperature used during the treatment process.

### Pre-Treatment and Bioreactors

The process of the invention employ a pre-treatment and processing system to hydrolyze cellulosic materials contained in the municipal solid waste. The methods and systems employ one or more pre-treatment reactors, and a biofuel/bioenergy biosynthetic system, which comprises one or more anaerobic fermentation and/or methanogenic bioreactors, and also one or more photosynthetic bioreactors. The invention basically involves the feeding of the pre-treated solid waste (as described above) into a bioreactor system and the operation mechanisms of the biosynthetic reactors in the production of biofuels.

The system generally comprises one or more reactors for pre-treating the cellulosic recalcitrant waste which will produce basic carbon sources to be used in the biosynthetic bioreactors. The system further comprises one or more bioreactors for fermentation or methanogenesis of the organic material contained in the treated solid waste, and in some embodiments, a photosynthetic bioreactor operating off of the CO₂ effluent as a carbon source. The pre-treated and biosynthesis systems may operate independently or be coupled to provide an integrated system.

The pre-treat reactors hydrolyze the recalcitrant organic waste including cellulose, hemicellulose, lignine, by action of one acid or alkaline compound. This process further results in the destruction of pathogenic microorganisms by low pH, as well digestion and standardization of the waste material making the resultant material suitable for anaerobic fermentation and/or methanogenesis. The pre-treated process may take place in small or high volumes (industrial scale) as described in greater detail herein. The type of reactors and internal designs may be selected on the basis of, for example, desired volume and/or retention time, as well as, and the desired flow and agitation systems.

For the pre-treatment processes to be efficient, and to maximize the waste hydrolysis within a very large volume (e.g., at a pilot and industrial scale as described herein), an important parameter is the supply of mixing. Thus, the reactor design should be sufficient to provide sufficient mixing. For example, the pre-treat system may comprise at least one continuous stirred-tank reactor or a similar reactor design with a sufficient mixing mechanism. Alternatively, or in addition, the pre-treat system may comprise a tubular recirculating reactor to perform mixing.

The pre-treat reactor general contains a mechanism for the mixing of solid waste. This reactor employs the action of chemical acid or alkali compounds including H₂SO₄ and NaOH at relatively high temperatures between 25 and 100° C. For example, in some embodiments, the hydrolysis of cellulosic materials including papers is accomplished at low pHs in the system in the range of from 1 to 3, during 1 to 8 hours.

In some other embodiments where recalcitrant cellulosic materials are present in high concentrations, such as from some plant peel or straw, the pre-treatment system may comprise an alkaline treatment using NaOH or Ca(OH)₂ in a continuous stirred tank reactor followed by a tubular reactor, such as a plug flow reactor. In this case the hydrolysis can be accomplished at high pHs in the range of from 10 to 12.

After the physicochemical treatment of the MSW cellulosic materials, the treated waste can be separated in degraded and non-degraded materials. For example, separation system may comprise a sieve or strainer for separating degraded and non-degraded biomass. The degraded material having its pH restored to neutral, and containing available carbon sources may flow from the pre-treating reactor to the bioreactor system for biofuel production. While non-degraded material may be added back to the biomass to provide and new pre-treating process.

Once the solid waste material is treated by the pre-treated system, e.g., significant amounts of recalcitrant waste was hydrolyzed, the treated waste (or available carbon source) having its pH restored to neutral or near neutral range (e.g., in the range of 5 to 7.5, or in the range of 6 to 7, or to about 6.5) is transferred or flows to a biosynthesis reactor to produce biofuel and bioenergy products. Generally, the biosynthesis system will comprise an anaerobic bioreactor for fermentation or methanogenesis. For example, the anaerobic bioreactor may be a UASB methanogenic (methane-producing) digester or an expanded granular sludge bed (EGSB) digester.

An Upflow Anaerobic Sludge Blanket (USAB) reactor uses an anaerobic process whilst forming a blanket of granular sludge which suspends in the tank. The treated sludge flows upwards through the blanket and is processed by the anaerobic microorganisms. The upward flow combined with the settling action of gravity suspends the blanket with the aid of flocculants. Small sludge granules begin to form whose surface area is covered in aggregations of bacteria. In embodiments having an absence of a support matrix, the flow conditions create a selective environment in which only those microorganisms, capable of attaching to each other, survive and proliferate. Eventually the aggregates form into dense pellet biofilms referred to as “granules.”

The UASB reactor is a high rate anaerobic system for sewage treatment and methane production. This reactor has been used also to digest the industrial contaminated effluents, including pharmaceutical, chemical, and food industries, as well as in the animal manure treatment. Therefore, this reactor may be efficiently used in the digestion of pre-treated home and municipal solid waste for the production of valuable biofuels and bioenergy products including ethanol, butanol and methane as described in U.S. Pat. Nos. 4,368,056; 7,015,028; and 7,556,737, which are hereby incorporated by references.

An Expanded Sludge Bed Digester (EGSB) reactor is a variant of the UASB-type reactor. The EGSB reactor allows for a faster rate of upward-flow for the wastewater passing through the sludge bed. The increased flux permits partial expansion (fluidization) of the granular sludge bed, improving wastewater-sludge contact as well as enhancing segregation of small inactive suspended particle from the sludge bed. The increased flow velocity is either accomplished by utilizing tall reactors, or by incorporating an effluent recycle (or both). This reactor also may be adapted, in the present invention, to the treatment of home and municipal solid waste.

Ethanol production may be performed in a fluidized bed recirculating tubular bioreactor containing a yeast (Saccharomyces sp.) or bacteria (Zymomonas sp.) attached and forming biofilms to suspended solid or liquid supports. The ethanol reactor may work independently, or may operate in series to receive biomass from the pre-treatment system, and to support one or more photosynthetic bioreactors by the effluent CO₂ from the anaerobic process. Some examples are described in U.S. Pat. Nos. 5,677,154; 5,779,164; 5,975,439 which are hereby incorporated by reference.

The biofuel synthesis system may also comprise at least one photosynthesis bioreactor, which may be a heterogeneous system containing photosynthetic microorganisms such as blue green algae and other microalgae species described herein, which may be supported by effluent CO₂ from the anaerobic bioreactors. Photo reactors are known, such as those described in U.S. Pat. No. 7,371,560, which is hereby incorporated by reference in its entirety. The aeration, temperature, pH, nutritional requirements, intensity and wavelength of light (e.g., white light), as well as duration of light/dark cycles suitable for photosynthetic microorganisms are known, for example, as also described in U.S. Pat. No. 7,371,560.

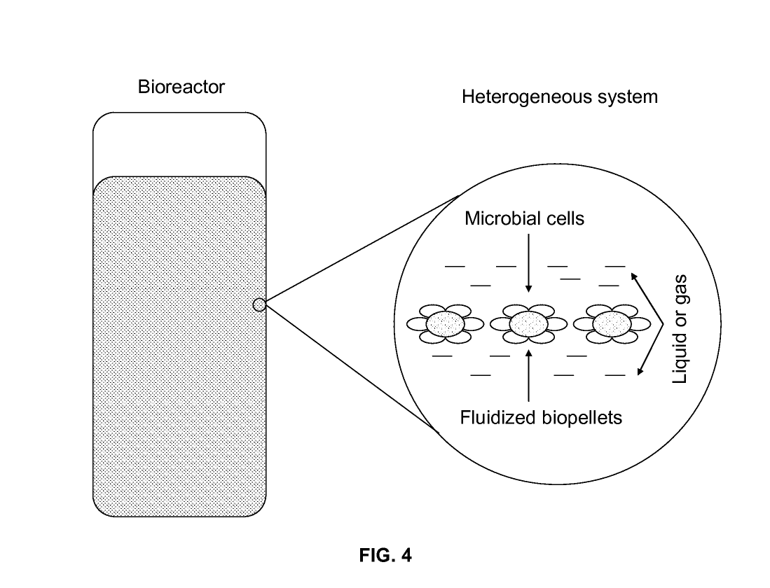

In some embodiments, the anaerobic bioreactors for synthesis of biofuels and bioenergy products may harness microbes in aqueous suspension and/or supported on surfaces. For example, the bioreactors may comprise solid surfaces that support biofuel-synthesizing microbes within biofilms. The solid surfaces may be composed of a variety of materials including porous glass, silicone rubber, as well as polymeric or metal surfaces among others. The solid surfaces may form a fixed or fluidized bed into the reactor (see FIG. 4). Alternatively or in addition, one or more support surfaces may be in the form of polymeric beads and the like, which may form a support bed or support matrix.

In some embodiments, the bioreactors for biosynthesis are heterogeneous reactors having solid phases (support surfaces and microbial cells), liquid phases (aqueous and/or organic phases), and gas phases (air, and gas produced by microbial metabolism). Aqueous liquid and gas phases may circulate within or between the various reactors as described herein. Where aqueous and organic (oil) phases are employed, microbes may form dropped biofilms at the liquid interface as well as on the solid support surfaces.

### Microorganisms

The microorganisms for biofuel synthesis may, for example, include one or a consortium of bacteria as methanogens, yeasts such as Saccharomyces sp., anaerobic bacteria such as Clostridium sp., or microalgae such as Botrycoccus sp. or Gloebacter sp., among others. Exemplary microorganisms having known biosynthesis activity are provided below (Table 1), together with exemplary enzymes involved in biosynthesis pathways. Examples of enzymes for synthesizing particular biofuel compounds are also provided. These enzymes may be genetically engineered to increase their performance.

Where methane is a desired biofuel product, at least one bioreactor for biosynthesis is an anaerobic reactor that comprises a methanogenic microorganism, which may include one or a consortium of Methanobacterium sp., Methanothrix sp., Methanosarcina sp., and Methanomonas sp. Other methanogenic microbes that may be used, and are described in U.S. Pat. No. 6,555,350, which is hereby incorporated by reference. For example, methanogens also include Methanococcus sp, Methanomicrobium sp., Methanospirilliam sp., Methanoplanus sp., Methanosphaera sp., Methanolobus sp., Methanoculleus sp., Methanosaeta sp., Methanopyrus sp., and/or Methanocorpusculum sp.

TABLE 1
Microorganisms and Enzymes for Biosynthesis of Biofuels and Bioenergy
Products
Methane

| | |
|---|---|
| Exemplary | Organic acids, CO2 |
| carbon | |
| sources | |
| Enzymes | formylmethanofuran dehydrogenase, |
| involved | methyltetrahydro-methanopterin: coenzyme M |
| | methyltransferase (Mtr), heterodisulfide |
| | reductase (Hdr), |
| | F₄₂₀H₂ oxidase (FprA), |
| | formaldehyde activating enzyme (Fae) |
| | methenyltetrahydromethanopterin cyclohydrolase, |
| | methylenetetrahydromethanopterin reductase, |
| Exemplary | Methanococcus sp, |
| Organisms | Methanomicrobium sp. |
| | Methanospirilliam sp. |
| | Methanoplanus sp. |
| | Methanosphaera sp. |
| | Methanolobus sp. |
| | Methanoculleus sp. |
| | Methanosaeta sp. |
| | Methanopyrus sp. |
| | Methanocorpusculum sp. |
| | Methanosarcina |

Ethanol

| | |
|---|---|
| Exemplary | Biomass, and carbonic metabolites produced |
| carbon | after xenobiotic biodegradation |
| sources | |
| Enzymes | Alcohol dehydrogenase (A, B, and C) |
| involved | Acetaldehyde dehydrogenase |
| | Amylases |
| | Glucoamylases |
| | Invertases |
| | Lactases |
| | Cellulases |
| | Hemicellulases |
| Exemplary | Saccharomyces sp. |
| Organisms | Klyveromyces sp. |
| | Zymomonas sp. |

Butanol

| | |
|---|---|
| Exemplary | Biomass, and carbonic metabolites produced |
| carbon | after xenobiotic biodegradation |
| sources | |
| Enzymes | Acetyl-CoAacetyltransferase |
| involved | Acetoacetyl-CoAthiolase |
| | 3-hydroxybutyryl-CoAdehydrogenase |
| | Crotonase |
| | Butyryl-CoAdehydrogenase |
| | Aldehyde/alcohol dehydrogenase |
| Exemplary | Clostridium sp. |
| Organisms | |

Hydrogen

| | |
|---|---|
| Exemplary | Water and light |
| sources | |
| Enzymes | Hydrogenases |
| involved | |
| Exemplary | Clostridium sp. |
| Organisms | |

Biodiesel

| | |
|---|---|
| Exemplary | CO2, and light |
| carbon source | |
| Enzymes | Lipasas (Triacylglycerolhydrolases) |
| involved | |
| Exemplary | Botrycoccus sp. |
| Organisms | Gloebacter sp |
| | Chlorella sp. |
| | Synechococcus sp. |
| | Synechocystis sp. |
| | Nitzchia sp. |
| | Schizochytriu sp. |

Methanol

| | |
|---|---|
| Exemplary | Methane and oxygen |
| carbon source | |
| Enzymes | Methane monooxygenase |
| involved | Formate dehydrogenase |
| | Formaldehyde dehydrogenase |
| Exemplary | Methylomonas sp. |
| Organisms | Methylosinus sp. |
| | Methylococcus sp. |

Some methanogenic species are highly thermophilic and thus can grow at temperatures in excess of 100° C. Where the methanogen is highly thermophilic, a separate (e.g., independent) bioreactor for synthesis of methane may be preferred. In certain embodiments, the methanogen is one or a consortium of Methanosarcina, Methanosaeta and/or Methanothrix species, which may carry out conversion of acetate and similar small-molecule carbon substrates to methane and carbon dioxide. Methanogens may use small organic compounds as substrate, such as formic acid (formate), methanol, methylamines, dimethyl sulfide, and methanethiol, which may be produced in the system.

The methanogenic bacteria may be naturally-selected for high methane producing activity, or alternatively, may be genetically modified by known techniques. The methanogenic enzymes that may be genetically engineered include, formylmethanofuran dehydrogenase, methyltetrahydro-methanopterin: coenzyme M methyltransferase (Mtr), heterodisulfide reductase (Hdr), F₄₂₀H₂ oxidase (FprA), formaldehyde activating enzyme (Fae), methenyltetrahydromethanopterin cyclohydrolase, and methylenetetrahydromethanopterin reductase. These enzymes have been isolated from species including, Methanococcus, Methanothermobacter, methanosarcina, methanopyrus, among others.

Biogas may be produced during anaerobic decomposition of the treated municipal solid waste. “Biogas” is a product of anaerobic digestion. In the absence of oxygen, anaerobic bacteria decompose organic matter and produce a gas mainly composed of methane (about 60%) and carbon dioxide. This gas can be compared to natural gas, which is approximately 99% methane. Biogas can be collected and used as an energy source for generators, boilers, burners, dryers or any equipment using propane, gas or diesel. Alternatively, methane may be recovered from the biogas as described below.

The process and systems described herein may be designed to produce alcohols, such as alcohols containing one to nine carbon atoms. Particular examples of alcohols that can be produced according to the invention include propanol, butanol, pentanol, hexanol, heptanol, octanol and nonanol. For the synthesis of alcohols, particularly ethanol, at least one bioreactor is an anaerobic reactor comprising fermentative microorganisms, such as one or more Zymomonas sp. and/or Saccharomyces sp. (e.g., Saccharomyces cerevisiae). Additional microorganisms suitable for fermentation of the biomass include a number of yeasts such as Klyveromyces sp., Candida sp., Pichia sp., Brettanomyces sp., and Hansenula sp. and Pachysolen sp. Alternatively, the microorganism may be one or a consortium of bacterial species such as Leuconostoc sp., Enterobacter sp., Klebsiella sp., Erwinia sp., Serratia sp., Lactobacillus sp., Lactococcus sp., Pediococcus sp., Clostridium sp., Acetobacter sp., Gluconobacter sp., Aspergillus sp., and Propionibactedum sp.

Various organisms for the production of fermentation products are known, as well as conditions for growth and substrate requirements, and are described for example, in U.S. Pat. No. 7,455,997, U.S. Pat. No. 7,351,559, U.S. Pat. No. 6,555,350, and U.S. Pat. No. 7,354,743, which descriptions are hereby incorporated by reference. In certain embodiments, the desired product is butanol, and the fermentative microorganisms include one or a consortium of bacteria including Clostridium sp.

The fermentative microorganisms, for example to produce ethanol, may be naturally selected for the production of the desired product, or may be genetically engineered to express desired enzymes. Techniques for genetic manipulation of bacteria and yeasts are well known, and include introduction of extrachromosomal elements by plasmid or phage, or integration of such elements into the host genome. Exemplary enzymes that may be genetically engineered include, Alcohol dehydrogenase (A, B, and C), Acetaldehyde dehydrogenase, Amylases, Glucoamylases, Invertases, Lactases, Cellulases, and Hemicellulases, among others.

In certain embodiments, the process and systems described herein will include a photo reactor to convert CO₂ produced during anaerobic biofuel synthesis to biofuel products such as hydrogen gas and lipids. Lipids may be employed in the production of biodiesels, for example, by trans-esterification. Photosynthetic organisms for use in the production of hydrogen gas and lipids from CO₂ are known, and include one or a consortium of naturally selected or genetically modified Synechococcus sp., Chlorella sp., Synechocystis sp., Nitzchia sp., Botrycoccus sp. and/or Schizochytriu sp., among others.

For the production of hydrogen, the method and system may employ photosynthetic microorganisms capable of using water as an indirect substrate for hydrogen production. Such microorganisms generally express one or more hydrogenases. These may include cyanobacteria and algae, such as green algae, blue-green algae, or red algae. Exemplary species include Synechococcus sp., Chlorococcales sp. Gloebacter sp. and Volvocales sp., among others.

The aeration, temperature, pH, nutritional requirements, intensity and wavelength of light (e.g., white light from natural or artificial light source), as well as duration of light/dark cycles suitable to support the growth and metabolism of photosynthetic microorganisms are known, for example, and are described in U.S. Pat. No. 7,371,560 which is hereby incorporated by reference.

### Operating Conditions

The physicochemical pre-treat reactors are filled with acid or alkali solution to hydrolyze the lignocellulosic materials of MSW. The process is performed in agitated conditions to mixing the materials, and at incubation temperatures chosen depending the amount and nature of materials. After the process, the hydrolyzed materials are passed to a deposit tank where the pH can be restored and/or food waste can be stoked. The available carbon sources from the hydrolyzed material or/and from soft food waste, are next flowed to the bioreactors to the further biosynthesis of biofuel products.

The bioreactor(s) are inoculated with the selected microorganism or mixed culture, followed by an acclimation period. The acclimation period may last one month, two weeks, one week, or less, during which biofilms, where present, are formed on support surfaces and the desired metabolic processes induced. Acclimation may involve, for example, induction or derepression of enzymes, multiplication of the initially small population(s) of bioleaching or synthetic microorganisms, selection for beneficial mutations, optimization of inorganic nutrients or other conditions, adaptation of microorganisms to toxins or inhibitors that may be present, and predation by certain protozoa.

Acclimation in some embodiments may proceed in steps, by first activating pre-treat reactor for input available carbon source material, followed by acclimating biosynthetic microorganisms for the production of the desired product(s). When using an integrated system as described herein, the flow of pre-treated waste to the anaerobic biosynthesis reactor can be controlled or initiated once predigested carbon source are available in pretreated system. Such embodiments may be useful particularly where the solid waste material contains components that are highly recalcitrants.

During the induction/acclimation period, it may be important to limit the concentration of the hydrolyzed carbon source and/or the flow of hydrolyzed source through the system. Growth and selection of biofuel-synthesizing microorganisms may be evaluated by the appearance and concentration of product being produced, as well as by the production of the expected metabolites (e.g., CO₂, methane, ethanol, butanol, hydrogen, etc.).

During acclimation and after acclimation, the bioreactor conditions may be adjusted as necessary to optimize product yield and rate of synthesis. Such conditions include hydrolyzed waste input concentration, flow rate, bioreactor temperature(s), levels of bioreactor agitation, pH, and levels of aeration or oxygenation. For example, the temperature of anaerobic reactors may be maintained within the range of about 15° C. to about 35° C., such as about 18 to about 32° C. The flow-rate of substrate through the system may depend on the volume of the bioreactor, the hydrolyzed concentration and biosynthesis rates, and may be maintained by a system of pumps and/or valves. The bioreactor may further allow for agitation of the liquid material.

After or continuously during the pre-treat process, the biomass and liquid hydrolyzed phase will be separated (e.g., by sieve, strainer or centrifugation). Upon efficient pre-treatment (hydrolyzed cellulose), the pH of the waste will be in the range of from about 1 to 3. After pH restored the cellulosic waste hydrolyzed can be used to biofuel synthesis (e.g., anaerobic fermentation or methanogenesis). For example, the pH of the biomass will generally be restored to near neutral (e.g., in the range of 5.0 to 7.5, such as about 6.5).

In certain embodiments where recalcitrant cellulosic waste are used an alkaline hydrolysation using NaOH or Ca(OH)₂ will be preferred. In this case and after the treatment the pH of hydrolyzed waste material will be in the range from about 10 to 12. In this case the pH of liquid phase may be restored with suitable acid including sulfuric acid.

In certain embodiments, complete pre-treatment of solid waste may take place first, independently of biofuel synthesis, or alternatively, biosynthesis may take place simultaneously with pre-treating in a coupled pre-treating/synthesis bioreactor. In certain embodiments that employ independent pre-treat and biosynthesis reactors, the pre-treat process may proceed for from about 3 hours to about 1 week, or about 10 hours to about 3 days, or in certain embodiments, for about 1, about 2, about 3, about 4, or about 5 days. The length of time needed for the bioprocesses will depend on several conditions, including, hydrolyzed waste input concentration, flow rate, volume of bioreactors, bioreactor temperature(s), and levels of bioreactor agitation.

In certain embodiments, biofuel or bioenergy production is performed at micro-refineries or pilot-refineries of domestic homes and restaurant scales, respectively. At these scales, a continuous or semi-continuous integrated pre-treatment/biosynthesis system may operate, such that from 2 to 10 kilos or 10 to 100 kilos of organic solid waste material are digested by day.

In certain embodiments, biofuel or bioenergy production is at industrial scale with a continuous or semi-continuous integrated bioleaching/biosynthesis system, such that from about 100 to about 100,000 kilos of solid waste substrate are digested per day. For example, about 500 to about 10,000 kilos of municipal solid waste may be processed and converted to biofuels in a period of about 24 hours to about 48 hours.

### Recovery of Biofuels

Biofuel products may be recovered and/or purified by known and commercially available methods and devices.

Alcohols such as ethanol, methanol, and/or butanol may be recovered from liquid material by molecular sieves, distillation, and/or other separation techniques. For example, ethanol can be concentrated by fractional distillation to about 90% or about 95% by weight. There are several methods available to further purify ethanol beyond the limits of distillation, and these include drying (e.g., with calcium oxide or rocksalt), the addition of small quantities of benzene or cyclohexane, molecular sieve, membrane, or by pressure reduction.

Product gas, for example, as produced by anaerobic metabolism or photosynthesis, may be processed to separate the methane and/or hydrogen components. Methane, hydrogen, or biogas may be drawn off from the system as pipeline gas.

In accordance with the invention, methane and/or hydrogen may be recovered as a biofuel product. Methane may be recovered and/or purified from biogas by known methods and systems which are commercially available, including membrane systems known for separating gases on the basis of different permeabilities. See, for example, U.S. Pat. No. 6,601,543, which is hereby incorporated by reference. Alternatively, various methods of adsorption may be used for separating methane and hydrogen.

Other ways of collecting biofuel products including centrifugation, temperature fractionalization, chromatographic methods and electrophoretic methods.

In certain embodiments, the biofuel recovery/purification components may be integrated into the system, for example, by connecting the respective device or apparatus to the gas or liquid effluents from the biosynthetic bioreactors. The purified biofuels and bioenergy products may be stoked in a separate container(s).

### Integrated Systems

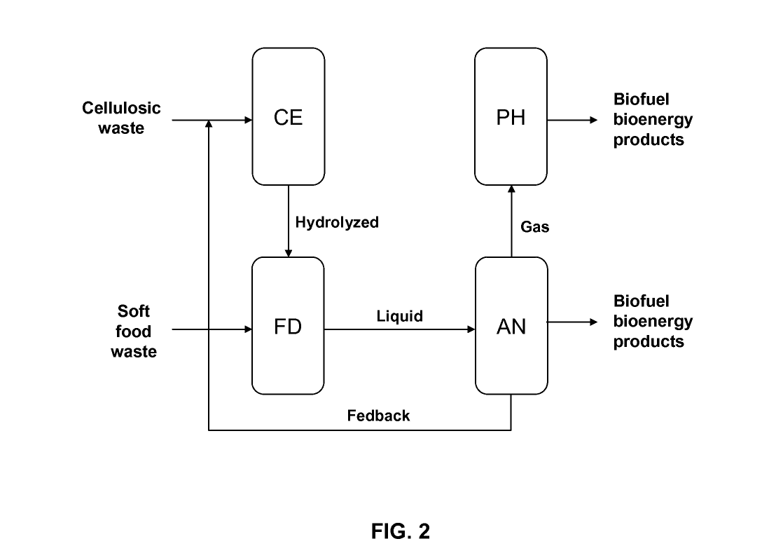

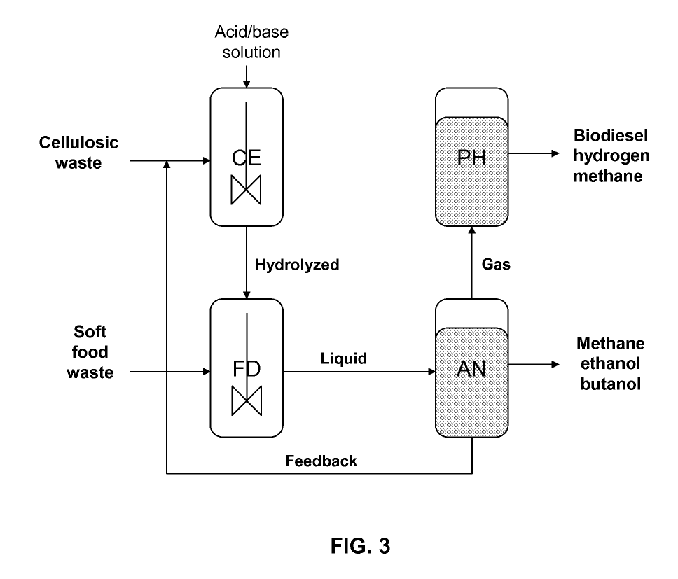

The present invention further provides an integrated in-series system for generating biofuels, such as hydrogen and methane, as well as other useful products such as ethanol, butanol, methanol, and biodiesel. These biofuels may be produced using as raw materials home and municipal solid waste. Exemplary integrated systems are illustrated in FIG. 2 and FIG. 3.

The integrated system basically comprises one pre-treated reactor suitable for hydrolyze the lignocellulosic materials and one connected deposit tank to stock the hydrolyzed material. In some embodiments, the deposit tank may contain only the hydrolyzed materials or only the soft food waste materials. In other embodiments, the deposit tank may contain both the hydrolysed material and the soft food waste. The deposit tank further contains an outlet for effluent liquid containing the carbon source to bioreactors.

The recipient system for pre-treat the lignocellulosic materials from MSW, comprises one or more like grinders, cuttings, or crushings for break down the large cellulosic materials. The recipient system may operate in series, between the pre-treating reactors and the anaerobic bioreactors, or may operate separately as an independent unit.

The pre-treating system may employ a continuous stirred-tank reactor or a recirculating reactor where the hydrolysis of lignocellulosic materials is performed. This hydrolysis reactor may be connected with a deposit tank containing only soft food waste, not needing a previously pre-treatment.

The integrated system further comprises an in-series anaerobic bioreactor, and optionally a photosynthesis bioreactor, to synthesize biofuels and bioenergy products from the treated lignocellulosic materials, or food waste. The anaerobic bioreactor contains an inlet for treated sludge from the pre-treated system, and an in-series connection to transport gas effluent from the biosynthesis anaerobic reactor to an algae photo bioreactor. The algae photo reactor may contain an inlet for liquid media containing necessary salts and inorganic nutrients (e.g., sea water or artificial medium having a similar chemical makeup). The anaerobic bioreactor system further comprises an outlet from the anaerobic biosynthesis bioreactor transporting the biofuel products, and an outlet from the photo bioreactor to transport the biofuel products.

In some embodiments, the anaerobic bioreactor may be a like upflow anaerobic sludge blanket UASB reactor or a like expanded granular sludge bed (EGSB) reactor for production of methane.

In some embodiments, the anaerobic and photo bioreactors internally may contain liquid surfaces (silicone oils) or solid surfaces (porous glass, silicone rubber, among others), where growing microorganisms may form pellet biofilms. The microorganisms growing in the anaerobic and photo bioreactors are mixed cultures of naturally selected or genetically engineered cells as described.

The system further comprises a feedback connection between the biosynthesis system to the pre-treated system to recycle all materials not completely degraded or used, to obtain total degradation of slurry materials flowing from bioreactors, and in this way to obtain a virtual “zero pollution system.”

A pump system may be employed for connecting inlets and outlets, to and from reactors and bioreactors, to maintain the desired flow through the system. Valves may also be present to allow for the flow of material through the system to be controlled.

The system may include sampling ports, so that concentrations of pre-treated MSW materials and/or biofuels produced can be monitored, as well as oxygen consumption, CO₂ production, pH, oxidation-reduction conditions, microbial cell physiology, biofilm health, among others.

The integrated system is generally a continuous system, with material circulating between the pre-treating and biosynthesis reactors, as controlled by the system of pumps. For example, in the continuous system, the anaerobic biosynthesis reactor may operate with an incoming flow rate equal to the outlet flow rate of liquid from the recipient unit or the pre-treating reactor (e.g., the flow rate of liquid from the first and/or second outlets of the bioleaching reactor). Further, the photo reactor may operate with a gas influent that is equivalent to the effluent gas of the anaerobic reactors.

In certain embodiments, as illustrated in FIG. 3, the integrated system is a coupled pre-treating system and a biosynthesis system. The bioreactor system for biofuels and bioenergy production comprises a heterogeneous anaerobic bioreactor for biosynthesis, and a heterogeneous photo bioreactor for biosynthesis. Biological structure of heterogeneous bioreactors has been described previously (see FIG. 4).

The working volume of the system may be from about 100 gallons to about 250 gallons in the house micro-refineries using waste food as mainly raw materials. However, the working volume of pilot refineries using waste food from restaurants could be from 250 gallons to 1000 gallons. While the industrial system may convert from about 1000 to about 10,000 gallons of liquid source material to useful product per run (in batch), or per day (when continuous). The operation or retention time for converting product to fuel may be between about 2 to 6 days, basically depending of the incubation temperatures.

In certain embodiments, the system is configured to allow for the transportation of treated cellulosic materials or waste food produced at one location (by a pre-treating reactor as described herein), to be transported to another location for biosynthesizing biofuel or bioenergy products (by bioreactors as described herein). Alternatively, the system may be entirely integrated to couple pre-treating system with the biosynthesis of bioenergy products from the treated material. The pre-treated system may be connected to, or positioned or located near, the production or source of home and MSW, so as to obviate the need to transport the waste for disposal. For example, the pre-treating reactor components may be located within about 1 mile or less of the production or source of the home and MSW.

In certain embodiments, the biofuel recovery/purification components may be integrated into the total system, for example, by connecting the respective and specific device or apparatus to the gas or liquid effluents from the biosynthetic bioreactors. These devices may be integrated in both at micro-refinery scale or at macro-refinery scale. The purified biofuels and bioenergy products may be stoked in a separate container(s).

### EXAMPLES

### Example 1

### Testing of a Pre-Treating Physicochemical Reactor for the Hydrolysis of Solid Cellulosic Waste Materials

This example demonstrates that recalcitrant lignocellulosic materials from home and municipal solid waste can be hydrolyzed in a pre-treat phisicochemical reactor. The MSW contain high amounts of lignocellulosic materials including paper, organic plastics, peels of food vegetables, and yard waste. These cellulosic materials are difficult to process directly in a biosynthetic bioreactor.

An alternative to physicochemical processes for breakdown (hydrolysis) these cellulosic materials is the acid or alkaline treatment at relative high temperatures. Physicochemical treatment in accordance with the invention has several advantages over other methods including enzymatic methods in terms of its simplicity economy, and high yield of cellulose hydrolysis.

The activity of acid or alkali compounds are enhanced with mixing and incubation processes at temperatures between 25 to 100° C. At high incubation temperatures the hydrolysis process may be more performant; however, these can be choice depending of the size of material waste to process.

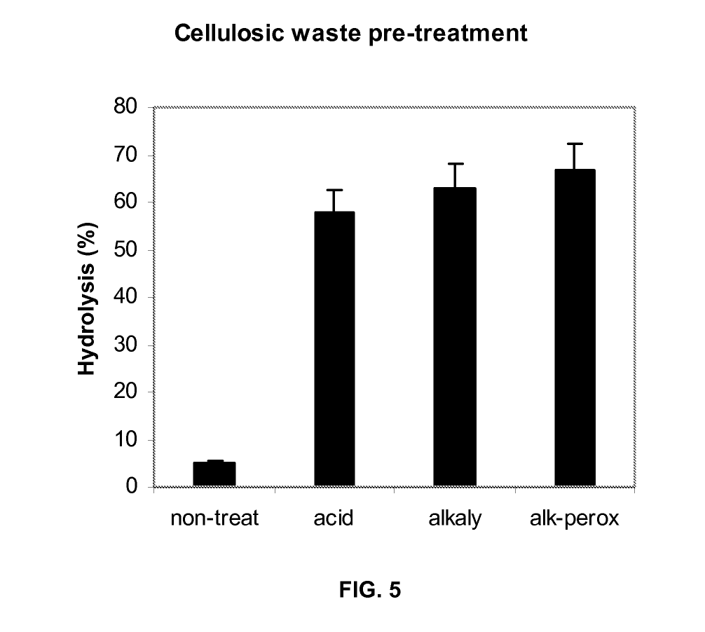

Fragments of cellulosic materials including paper, cardboards, and organic plastics were placed in a 1-L reactor containing one of these compounds: H2SO4, or NaOH, or NaOH/H2O2. Reactor was slowly agitated to mixing and maintained at 35° C. during 8 hours. After the treatment, samples were taken form the reactor to analyze the hydrolysis performance and accumulation of sugars. As is shown in FIG. 5, after an 8-hours incubation period the hydrolysis using acid, alkali and alkali/peroxide reached until 58, 63, and 67%, respectively. In contrast, cellulose hydrolysis in a control system (without physicochemical treatment) was only around 4%.

These results demonstrate that the pre-treating reactor was highly efficient in the hydrolysis of cellulosic materials, including paper, cardboard, and plastics. This process can be used efficiently at laboratory, pilot and industrial level to hydrolyze cellulosic materials from MSW, to thereby condition the MSW for production of biofuels.

### Example 2

### Testing of a Heterogeneous Bioreactor for the Production of Methane from Treated Cellulosic Waste Material

This example shows that the pre-treated MSW (cellulose hydrolyzed) can be efficiently metabolized to produce methane in a bioreactor. The aim of physicochemical treatment is to improve or allow the anaerobic digestion of the solid waste, where high concentrations of cellulosic materials are present.

Batch tests were performed in a 1-L UASB anaerobic reactor to assess waste biodegradability and production of methane. The reactor was fed with waste previously treated to hydrolyze the cellulose components. A bacteria consortium of a previous methanogenic bioreactor was used as inoculum. Treated cellulosic waste, non-treated cellulosic waste, soft food waste, and a control sample consisting of ethanol, were digested. The total organic processed for each matter was equivalent to 10 g of chemical oxygen demand (COD)/L in all samples.

The biogas volume produced was measured by movement of liquid (water, pH 2, NaCl 10%). Biodegradability was assessed by comparison of biogas volumes produced by treated, untreated and control samples. The biodegradable percentage was estimated by comparing the biogas volume produced with sludge (treated or untreated) to the biogas volume produced with control.

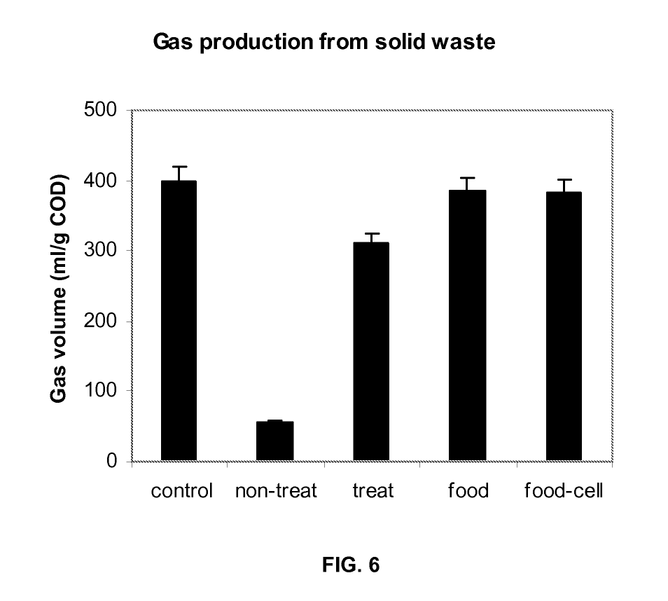

The results of FIG. 6 shows that at the hydraulic retention time of 4 to 8 hours, approximately 310, 386, and 382 ml of biogas/gram of COD were produced from treated, non-treated, and half-treated, respectively. These results were compared with positive (ethanol) and negative (non-cellulose hydrolyzed) controls where around 400 and 56 ml gas/g of COD, respectively.

These results indicate that MSW treated to hydrolyze cellulosic materials allows higher biogas production than with untreated MSW, and close to an ethanol control. These results also demonstrate that the physicochemical treatment is efficient in the hydrolysis of cellulosic materials, conducting to a good performance of methanogenic bioreactors.

### Example 3

### Testing of a Heterogeneous Bioreactor System for the Production of Ethanol from Treated Cellulosic Waste Material

This example demonstrates the production of ethanol from treated (hydrolysis of cellulosic materials) municipal solid waste.

A 1-L bioreactor containing 500 ml of pre-treated waste (10 g COD/L) was inoculated with a selected culture of the yeast of Saccharomyces sp. A control system containing glucose as carbon source was used to compare with treated and non-treated lignocelluloic samples. The reactor was maintained at room temperature and agitated only by recirculation. Samples were collected and analyzed every 8 hours for 3 days for COD and ethanol concentrations.

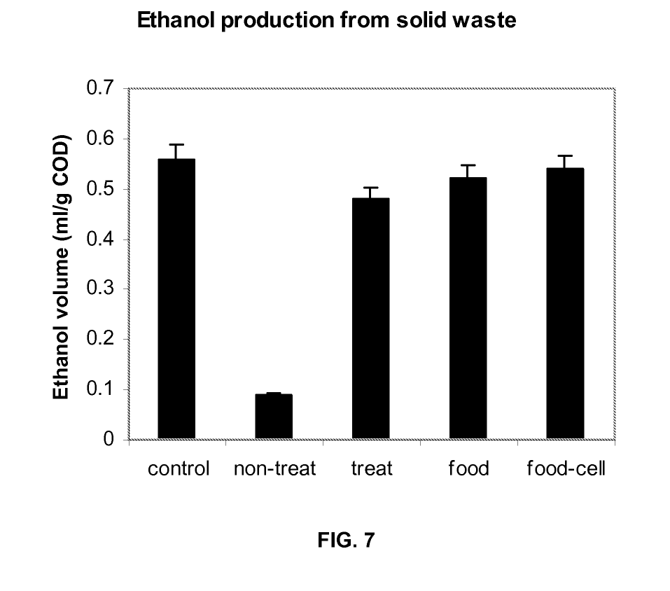

The results show that ethanol reached its maximum production after 48 hours of incubation after which production decreased. As can be observed in FIG. 7, after 48 hours of culture, approximately 0.48, 0.52, and 0.54 ml of ethanol/gram of COD were produced from treated, non-treated, and half-treated, respectively. These results were compared with positive (glucose) and negative (non-cellulose hydrolyzed) controls where around 0.56 and 0.09 ml ethanol/g of COD, respectively.

These results indicate that the pre-treatment of municipal solid waste to hydrolyze the cellulosic materials increases significantly the production of ethanol from the solid waste. The results suggest that this ethanol production process from treated solid waste could be efficiently performed at laboratory, pilot and industrial scales.

### Example 4

### Testing of a Photo Bioreactor for the Production of Hydrogen and Biodiesel from the CO₂ Effluents

This example demonstrates that biodiesel and hydrogen are produced by microalgae cultures extracting the CO₂ from the effluent of the anaerobic bioreactors.

Cultures of microalgaes (Botrycoccus sp. and Gloebacter sp.) were used to assess cell-lipid accumulation and hydrogen production, respectively, after capture of different CO₂ concentrations produced in anaerobic bioreactors. Both but separately algae cells were grown in a 1-L bioreactor culture 16 hs light followed by 8 hs dark with a fluorescent lamp (15 Wm-2) at 28° C. The photobioreactor was bubbled with effluent gas from anaerobic bioreactor to reach 0.5 and 1.0% (v/v) levels of CO₂. The growth of culture was recorded at 2-day intervals.

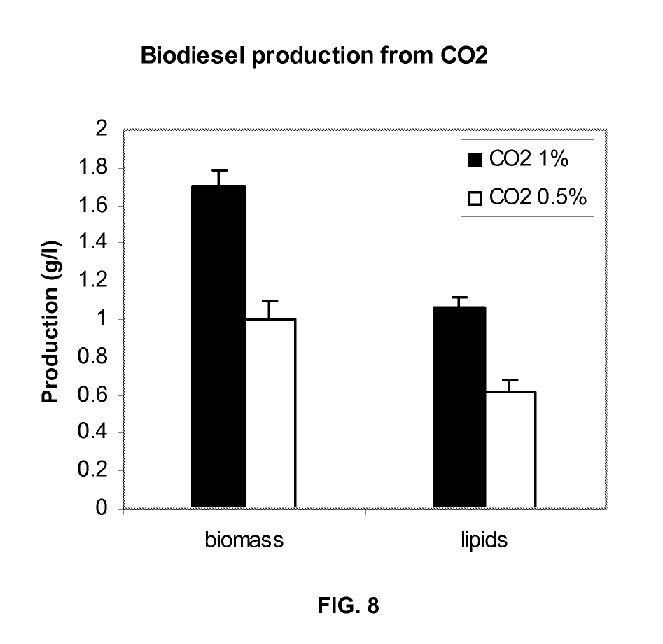

Botrycoccus sp. cells were harvested at the late logarithmic growth phase, collected and evaluated for biomass accumulation, and lipid content. FIG. 8 shows the performance of a biosynthetic multiphasic photosynthetic reactor in the production of biodiesel from CO₂ outlet from an anaerobic bioreactor. Performance was assessed by the yields of biomass according to the concentration of CO₂ injected. Substantial biomass is achieved at 1% and 0.5% CO₂ concentrations, with more biomass produced at 1%. The lipids extracted to produce biodiesel reached approximately 61% of the biomass produced at different concentration of CO₂.

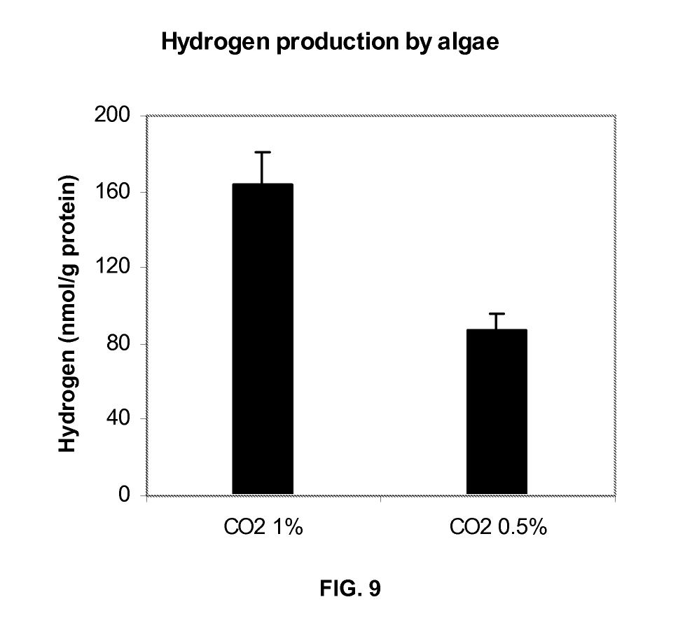

FIG. 9 shows the performance of Gloebacter sp. cultures with respect to the production of hydrogen in relation to biomass (nmol hydrogen per gram of protein). The hydrogen release was approximately 164 nmol of hydrogen per gram of protein when biomass was grown at 1% of CO₂, while 87 nmol of hydrogen per gram of protein was produced when 0.5% CO₂ was injected.

These results demonstrate that the CO₂ effluent from a municipal solid waste-digesting anaerobic bioreactor can be captured and fixed to support the growing of microalgae cells in a photo bioreactor system. This photo bioreactor can be used efficiently to capture the CO₂ produced in the anaerobic reactors, before that CO₂ is disposed in the environment.

The foregoing description of some specific embodiments provides sufficient information that others can, by applying current knowledge, readily modify or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. In the drawings and the description, there have been disclosed exemplary embodiments and, although specific terms may have been employed, they are unless otherwise stated used in a generic and descriptive sense only and not for purposes of limitation, the scope of the claims therefore not being so limited. Moreover, one skilled in the art will appreciate that certain steps of the methods discussed herein may be sequenced in alternative order or steps may be combined. Therefore, it is intended that the appended claims not be limited to the particular embodiment disclosed herein.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference. More generally, documents or references are cited in this text, either in a Reference List before the claims; or in the text itself; and, each of these documents or references (“herein-cited references”), as well as each document or reference cited in each of the herein-cited references (including any manufacturer's specifications, instructions, etc.), is hereby expressly incorporated herein by reference.

### REFERENCES

Ascon M A, Lebeault J M. (1999). High Efficiency of a coupled aerobic-anaerobic recycling biofilm reactor system in the degradation of recalcitrant chloroaromatic xenobiotic compounds. Appl. Microbiol. Biotechnol. 52(4):592-9.
Ascon M A, Thomas D, Lebeault J M. (1995). Activity of synchronized cells of a steady state biofilm recirculated reactor during xenobiotic biodegradation. Appl. Environ. Microbiol. 61(3): 920-5.
Ascon M A, Ascon D B, Lebeault J M. (1995). Degradation activity of adhered and suspended Pseudomonas cells cultured on 2,4,6-trichlorophenol, measured by indirect conductimetry. J Appl Bacteriol. 79:617-624.
Ilker U z, M E Rasche, T Townsend, A V Ogram, and A S Lindner. (2003). Characterization of methanogenic and methanotrophic assemblages in landfill samples. Proc Biol Sci. 270: S202-S205.
Murphy, J D and McKeogh, E. (2004). Technical, economic and environmental analysis of energy production from municipal solid waste. Renew Energ. 29: 1043-1057.
Vinzant T B, Adney W S, Grohmann, K, and Rivard C J. (1990). Aerobic and anaerobic digestion of processed municipal solid waste effects of retention time on cellulose degradation. Appl Bioche and Biotechnol. 24/25:765-771
Rivard, C J, Himmel M E, Vinzant T B, Adney W S, Wyman C E, and Grohman K. (1990). Anaerobic digestion of processed municipal solid waste using a novel high solids reactor: maximum solids levels and mixing requirements. Biotechnol. Lett. 12: 235-240.
Owens J M and Chynoweth. (1993). Biochemical methane potential of municipal solid waste (MSW) components. Wat. Sci. Tech. 27: 1-14.
Karakashev D, Batstone D J, and Angelidaki I. (2005). Influence of environmental conditions on methanogenic compositions in anaerobic biogas reactors. Appl Environ Microbiol. 71: 331-338.
Liu D, Liu D, Zeng R J, Angelidaki I. (2006). Hydrogen and methane production from household solid waste in the two-stage fermentation process. Water Res. 40: 2230-2236.
Wu M C, Sun K W, and Zhang Y. (2006). Influence of temperature fluctuation on thermophilic anaerobic digestion of municipal organic solid waste. J Zhejiang Univ Sci B. 7: 180-185.
Taherzadeh M J and Karimi K. (2008). Pretreatment of Lignocellulosic Wastes to Improve Ethanol and Biogas Production: A Review. Int J Mol. Sci. 9: 1621-1651.

1. A method for producing biofuel or bioenergy product(s) from home or municipal organic solid waste, comprising:
hydrolyzed cellulose materials from the home and municipal solid waste through the action of acid or alkali treatment, to thereby produce a treated and available carbon sources; and
synthesizing one or more biofuel or bioenergy products in bioreactors by microbial action using as carbon source the previously hydrolyzed waste materials

2. The method of claim 1, wherein the solid waste material is all organic waste material generated at home or municipalities.

3. The method of claim 2, wherein the organic solid waste includes mainly the lignocellulosic materials.

4. The method of claim 3, wherein the cellulose materials include also hemicellulose, and lignine materials.

5. The method of claim 1, wherein the solid waste include also the food waste from homes and restaurants.

6. The method of claim 5, wherein the lignocellulosic materials are treated with a solution of acid or alkali compounds.

7. The method of claim 6, wherein the chemical solution comprises the H₂SO₄ (between 2 to 4% p/v) to hydrolyze the cellulose materials.

8. The method of claim 6, wherein the chemical solution comprises the NaOH or Ca(OH)₂ (between 1 to 6% p/v) to hydrolyze the recalcitrant cellulose materials.

9. The method of claim 8, wherein the hydrolyzed process include the increase of temperatures between 25 to 100° C. to increase the hydrolyze process.

10. The method of claim 9, wherein the method operates in batch, semi-continuously, or continuously.

11. The method of claim 10, wherein the pre-treat process takes place in a system comprising at least one agitated tank reactor or a tubular recirculated reactor

12. The method of claim 11, wherein the pre-treated system obtains a acid hydrolyzed waste pH of from 1 to 3, or alkali hydrolyzed waste pH of from 9 to 11.

13. The method of claim 12, further comprising, separating the hydrolyzed biomass using a sieve or strainer after the treatment when the pH is acid or alkaline

14. The method of claim 13, wherein the hydrolyzed material together with the soft food waste is stoked in a deposit tank.

15. The method of claim 14, further comprising, restoring the neutral pH in the deposit tank containing the available carbon source to fed the biosynthetic reactors.

16. The method of claim 15, further comprising, feeding back the solid not hydrolyzed waste for a new pre-treatment process.

17. The method of claim 16, wherein the biofuel or bioenergy product is methane, hydrogen, methanol, ethanol, butanol, and/or biodiesel.

18. The method of claim 17, wherein the synthesis of biofuels takes place in at least one anaerobic reactor.

19. The method of claim 18, wherein the anaerobic reactor is a heterogeneous bioreactor for production of methane.

20. The method of claim 19, wherein the anaerobic bioreactor is a like USAB reactor of an EGSB reactor.

21. The method of claim 18, wherein the anaerobic bioreactor is a heterogeneous bioreactor for the production of ethanol, butanol, or methanol.

22. The method of claim 21, wherein biofuel synthesis takes place in at least one photosynthesis bioreactor.

23. The method of claim 22, wherein the photosynthesis bioreactor is a heterogeneous bioreactor having algae or micro-algae cells forming biofilms

24. The method of claim 23, wherein the photosynthetic organisms are supported by effluent CO₂ from the anaerobic bioreactor.

25. The method of claim 24, wherein the microorganisms producing the biofuel or bioenergy product are listed in Table 1.

26. The method of claim 25, wherein the biofuel or bioenergy product is methane, and the methanogenic microorganisms are one or a consortium of Methanosarcina, Methanosaeta and/or Methanothrix species.

27. The method of claim 26, wherein the biofuel or bioenergy product is an alcohol, and the microorganisms include one or more Zymomonas sp. and/or Saccharomyces sp.

28. The method of claim 26, wherein the biofuel or bioenergy product is butanol, and the fermentative microorganisms include a Clostridium sp.

29. The method of claim 26, wherein the photosynthesis bioreactor includes one or a consortium of Botrycoccus sp., Gloebacter sp., Synechococcus sp., Chlorella sp., Synechocystis sp., Nitzchia sp., and/or Schizochytriu sp.

30. The method of claim 29, wherein the total process is performed at home in a micro-refinery scale.

31. The method of claim 29, wherein the method is performed at pilot or industrial scale.

32. The method of claim 31, further comprising, recovering the biofuel or bioenergy products.

33. The method of claim 32, wherein the biofuel or bioenergy product is ethanol, methanol, and/or butanol, and the product is recovered from liquid material by a molecular sieve or distillation.

34. The method of claim 33, wherein biogas containing hydrogen and/or methane is drawn off from the system as pipeline gas.

35. The method of claim 34, wherein hydrogen and methane are purified from biogas.

36. An integrated process (or refinery) system for converting home or municipal solid waste to one or more biofuel or bioenergy products, comprising:
a pre-treated system suitable for hydrolyze the cellulosic materials to obtain carbon sources easily to metabolism.
a anaerobic bioreactor operatively connected to receive said pre-treated hydrolyzed waste; and
a photo bioreactor operatively connected to receive gas effluent from the anaerobic bioreactor.

37. The integrated system of claim 36, wherein the pre-treat reactor contains an inlet for influent solid waste materials.

38. The integrated system of claim 37, wherein the pre-treated system contains a first outlet for hydrolyzed materials flowing to a deposit tank.

39. The integrated system of claim 38, wherein the pre-treated system comprises a agitated tank reactor.

40. The integrated system of claim 39, wherein the agitated tank reactor for cellulose treatment is followed by a deposit tank for hydrolyzed and food waste.

41. The integrated system of claims 36 to 40, wherein the anaerobic bioreactor is a UASB reactor or a EGSB reactor.

42. The integrated system of claim 41, wherein the anaerobic bioreactor is a heterogeneous bioreactor containing fermentative or methanogenic microbes forming biofilms.

43. The integrated system of claim 42, wherein the anaerobic bioreactor contains an inlet for treated waste from the pre-treat system.

44. The integrated system of claim 43, wherein the anaerobic bioreactor contains an in-series connection to transport gas effluent from the anaerobic reactor to the photo bioreactor.

45. The integrated system of claim 44, wherein the photo bioreactor contains an inlet for liquid media.

46. The integrated system of claim 45, wherein the anaerobic bioreactor further comprises an outlet for transporting the biofuels and bioenergy products.

47. The integrated system of claim 46, further comprising a feedback connection between the biosynthetic bioreactors to the pre-treating reactor.

48. The integrated system of claim 47, further comprising a system of pumps and/or valves operably connecting inlets and outlets.

49. The integrated system of claim 48, wherein the working volume of a home micro-refinery system is from about 10 gallons to about 100 gallons.

50. The integrated system of claim 48, wherein the working volume of the pilot and industrial system is from about 100 gallons to about 100,000 gallons.
